(19) United States
(12) Patent Application Publication
(10) Pub. No.: US 2005/0043708 A1
(43) Pub. Date: Feb. 24, 2005

(54) Anastomosis device and method

(76) Inventors: James B Gleeson, Columbus, OH (US); Herbert S Bresler, Bexley, OH (US); Brian C Kelley, New Albany, OH (US)

Correspondence Address: Richard M. Klein;Fay, Sharpe, Fagan, Minnich & McKee, 1100 Superior Avenue, 7th Floor, Cleveland, OH, 44114-2518, US

(21) Appl. No.: 10/503,287

(22) PCT Filed: Jan. 31, 2003
(86) PCT No.: PCT/US03/02929

Related U.S. Application Data
(60) Provisional application No. 60/353,973, filed on Jan. 31, 2002.

(51) Int. Cl. A61M 31/00

(52) U.S. Cl. 604/507; 604/506; 604/500

(57) ABSTRACT

An apparatus for treating a heart includes a housing member having a plurality of elongate channels defined therein and is movable between a first collapsed position and a second expanded position. A tissue attachment member is positioned in each channel. In one aspect, the apparatus is adapted for performing an anastomosis and includes a housing member having a plurality of elongate channels defined therein and which is movable between a fist collapsed position and a second expanded position. A surgical clip is positioned in each channel. A clip deployment mechanism projects the clips from their respective housings and a registration member approximates and aligns the first and second tubular structures. In a further aspect, helical barbs are movably affixed within sleeves formed on a sheetg of biocompatible material adapted for placement within a heart ventricle. Methods for treating a heart and reducing the volume of a heart ventricle are also provided. In a still further aspect, helical barbs provide attachment for patches for closure of a wound site or for suturing of a site needing closure. In yet another aspect of the invention, the helical barbs provide an attachment regine for implantable devices, as a well as a means to deliver medically efficacious materials to chosen sites with secure means. The helical device of the present invention further serves as a stent.

### FIELD OF THE INVENTION

The present invention relates generally to the art of surgical instruments and methods and, in particular, to an improved apparatus and method for performing an anastomosis or surgical connection between tubular structures or vessels. The present invention finds particular application in the anastomotic joining of vascular tissue for the purpose of bypassing an occluded or diseased section of a blood vessel, such as a coronary artery, and will be described with particular reference thereto. However, it will be recognized that the present invention is amenable to anastomosis in general. The present invention further has broader applications in many different environments, such as securing patches for the noninvasive correction of intestinal perforations, deep hole wound closures, and others. The present invention has still further applications in the medical field, such as in affixing medical devices to tissues, attaching biological microelectromechanical systems (MEMS), drug delivery, sensor attachment and uses as a stent.

### BACKGROUND OF THE INVENTION

Commonly in coronary artery bypass graft (CABG) procedures, one or more graft vessels are hand sutured into place between a blood source, such as the aorta, and a target coronary artery, such as the left anterior descending artery. Most CABG procedures are accomplished by opening the chest wall to gain access to the coronary vessels. Through the use of heart-lung bypass machines and cardioplegia to protect the heart, the heart is stopped to enable the surgeon to perform the precise manipulations required to hand suture the tiny, delicate vessels.

Although bypass grafting has been highly successful, there exists a need for minimally invasive techniques for bypassing the coronary arteries and for performing the anastomosis on a beating heart. Minimally invasive procedures have been developed in which the bypass is performed through a small incision in the chest wall. A number of techniques are also known for reducing the effects of vessel movement when performing the suturing on a beating heart. However, techniques that dampen or isolate the translation of movement from the beating heart to the artery can damage the vessel or cause myocardial injury.

Additionally, techniques are also known which rely on cooling the patient to slow the rate of the beating heart. This allows the surgeon to place the sutures between heartbeats. However, such techniques can increase the time it takes to perform the procedure and do not eliminate the movement of the artery.

Consequently, there is a need for a catheter-based, mechanical method for automating an anastomosis, i.e. the surgical connection of tubular. structures. Such an apparatus and method do not require hand suturing and provides for a leak-free connection between vesicles.

Separate and apart from the above, left ventricular enlargement or “remodeling” is a pathologic, progressive process that can follow myocardial infarction and other cardiomyopathies. The infarcted region becomes noncontractile and akinetic or dyskinetic, thus reducing the volume output of the heart. As a result, left ventricular enlargement occurs to restore or maintain output of oxygenated blood to the body. This dilation has the deleterious effect, however, of imposing an extra workload on the remaining healthy heart tissue and increasing wall tension, which, in turn, stimulates hypertrophy. With damage to the myocardium, however, these increased requirements placed on the contracting myocardium may be of such an extent that cardiac output requirements are not met, and the heart continues to dilate progressively. This cycle can lead to congestive heart failure, which is a major cause of death and disability in the United States.

Additionally, postinfarction left ventricular aneurysm is an extreme example of adverse left ventricular remodeling. Such an aneurysm leads to deterioration of cardiac functions and symptoms of congestive heart failure.

In order to address these difficulties, it is known to place a patch within an enlarged left ventricle to reduce the volume, improve ejection fraction, reduce wall stress, and otherwise to restore the ventricle to a more physiologic morphology and function. Typically, these procedures have required incising and introducing the patch through the heart wall and hand suturing the patch in place. Thus, there also exists a need for an endoventricular patch plasty apparatus and method that is catheter-based and that does not require hand suturing.

The present invention contemplates new and improved catheter-based tissue attachment devices and non-invasive methods which overcome the above-referenced problems and others.

### SUMMARY OF THE INVENTION

In a first aspect of the present invention, a catheter-based apparatus for treating a heart includes a housing member having a plurality of elongate channels defined therein and which is movable between a first collapsed position and a second expanded position. A tissue attachment member is positioned in each channel.

In a second aspect of the present invention, a device for performing an anastomosis between a first tubular structure and a second tubular structure includes a housing member having a plurality of elongate channels defined therein and which is movable between a first collapsed position and a second expanded position. A surgical clip is positioned in each channel. A clip deployment mechanism projects the clips from their respective housings and a registration member approximates and aligns the first and second tubular structures.

In a third aspect, an apparatus for altering the morphology of a heart includes a sheet of biocompatible material adapted for placement within a ventricle and having a plurality of generally rigid elongate sleeves attached thereto. The sleeves are spaced apart and extend radially. A surgical barb is movably secured within each sleeve. The barb moves between a first position in which the barb is constrained within its respective sleeve and a second position adapted for securing the sheet to an interior wall of the heart.

For example, this aspect of the present invention can be used for the treatment of the enlargement of the left ventricle that results from a variety of heart ailments. This condition leads to congestive heart failure with a median population mortality of 5 years. The present invention can be utilized to reduce the volume of the left ventricle in order to reduce stress in the myocardium and increase the ejection fraction of the heart. In utilizing this aspect, a diaphragm can be deployed by a catheter into the left ventricle of the heart, creating two separate chambers and reducing the overall volume.

In a fourth aspect, a method for treating a heart includes forming a first elongate incision in a vascular graft and a second elongate incision in a target coronary artery of the heart to define an anastomotic site. A catheter is inserted into the graft and the incisions in the graft and the target artery are aligned. A registration device is passed from the catheter through the first and second incisions, into the target artery, and a tissue-fastening device is passed from the catheter into graft. The tissue fastening apparatus includes a housing member having a plurality of elongate channels defined therein, the housing member being movable between a first collapsed position and a second expanded position, a surgical clip positioned in each channel, and a clip deployment mechanism for projecting the clips from their respective channels. The graft and target artery are approximated with the registration device and the clips are deployed from their respective channels. The housing member, clip deployment mechanism, registration device, and catheter are then removed from the anastomotic site.

In a fifth aspect of the present invention, a method for reducing the volume of a heart ventricle includes introducing a patch into the ventricle and securing the patch to an interior wall of the ventricle using barbs. The patch includes a sheet of biocompatible material adapted for placement within a ventricle, a plurality of generally rigid, elongate, and radially extending sleeves attached to the sheet, and a surgical barb movably secured within each sleeve.

The present invention is adapted to minimally invasive techniques, thus reducing the trauma, risks, recovery time, and pain that accompany current open-chest techniques.

Still further advantages and benefits of the present invention will become apparent to those of ordinary skill in the art upon reading and understanding the following detailed description of the preferred embodiments.

### BRIEF DESCRIPTION OF THE DRAWINGS

The invention may take form in various components and arrangements of components, and in various steps and arrangements of steps. The drawings, in which like reference numerals denote like components throughout the several views, are only for purposes of illustrating preferred embodiments and are not to be construed as limiting the invention.

FIG. 1 illustrates exemplary tubular structures with aligned incisions for anastomosis in accordance with the present invention.

FIG. 2 is a longitudinal cross-sectional view of portions of the graft and target vessels and shows a catheter device for intraluminally carrying and delivering the anastomosis device of the present invention.

FIG. 3 is a longitudinal cross-sectional view of the catheter sheathing identified in FIG. 2 and shows the housing storing the clips and the registration device.

FIG. 4 is a cross-sectional view taken along the lines 4-4 of FIG. 3.

FIG. 5 is a longitudinal cross-sectional view of the vessels and shows the housing storing the clips and the registration device deployed to approximate and align the incisions.

FIG. 6 is a view similar to that of FIG. 5, illustrating the position of the clips in a partially deployed state.

FIG. 7A illustrates the removal of the registration device and catheter after the clips are in place.

FIGS. 7B and 7C illustrate the preferred helical shape of the fastening clips, helical barbs and helical devices of FIGS. 7-11 when in use. FIG. 7B is a view of the helical shape from one side, and FIG. 7C is a side view of FIG. 7B.

FIG. 8 is a perspective sectional view of the completed anastomosis.

FIGS. 9 and 10 illustrate an alternative embodiment of the present invention in which the clips remain attached to the housing.

FIG. 11 illustratively shows a device attached in accordance with the present invention.

FIG. 12 illustrates the use of the helical device of the present invention as a stent.

### DETAILED DESCRIPTION OF THE PREFERRED EMBODIMENTS

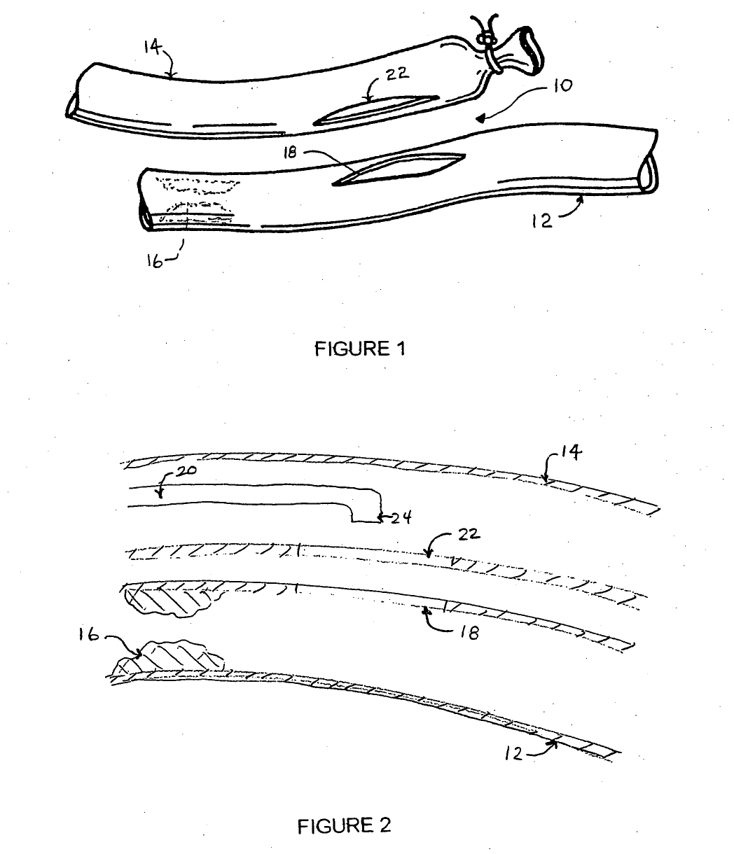

Turning now to the drawings, wherein the showings are for purposes of illustrating the preferred embodiments of the invention only and not for limiting the same, FIG. 1 shows an anastomotic site 10 involving a first vessel such as an artery 12 and a second vessel such as a graft 14. The artery 12 may be, for example, a coronary artery containing a stenosis 16 and the graft 14 may be, for example, a harvested vein or artery, or a synthetic vascular graft material such as expanded polytetrafluoroethylene (ePTFE) or the like. The location of one or more anastomotic sites are selected to bypass the blockage 16 and restore a physiologic blood flow to the areas downstream therefrom.

A longitudinal incision 18 is defined in the artery 12 at the intended anastomotic site 10, for example, by surgically exposing or accessing the artery and cutting. Alternatively, a blade or other means for forming the incision 18 can be advanced to the anastomotic site 10 intraluminally in a catheter via the artery 12 for forming the incision 18 percutaneously. A corresponding incision 22 is also defined in the graft 14.

The graft vessel 14 can be a harvested blood vessel segment such as the saphenous vein or interior mammary artery (IMA), or, a synthetic vascular graft material. Alternatively, the graft 14 can be a nearby vessel anastomosed to the artery 12 in situ. In still other embodiments, the graft 14 may be a vessel, such as the IMA, that is harvested at one end only and left attached at the end distal to the anastomotic site.

Referring now to FIG. 2, there appears in the graft 14 a catheter 20 having an end 24 distal to the operator (not shown). The catheter 20 defines a channel through which the anastomosis device of the present invention is passed.

In operation, the graft 14 and artery 12 are placed in longitudinal alignment so that the respective incisions 22 and 18 are substantially in aligned, facing relation. The catheter is then advanced within the lumen of the graft 14 until the distal end 24 is aligned with the incision 22.

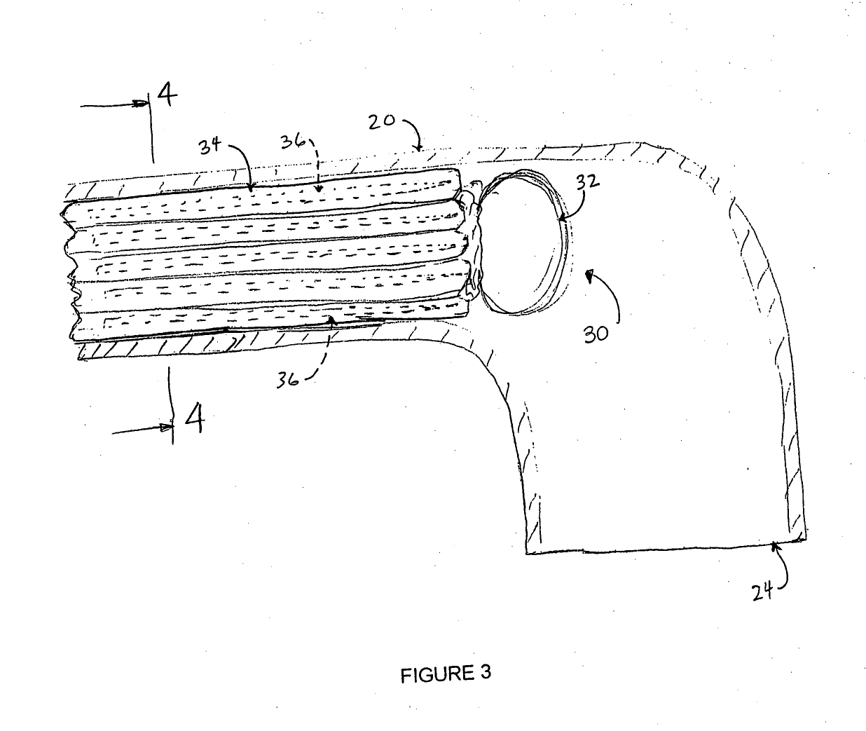

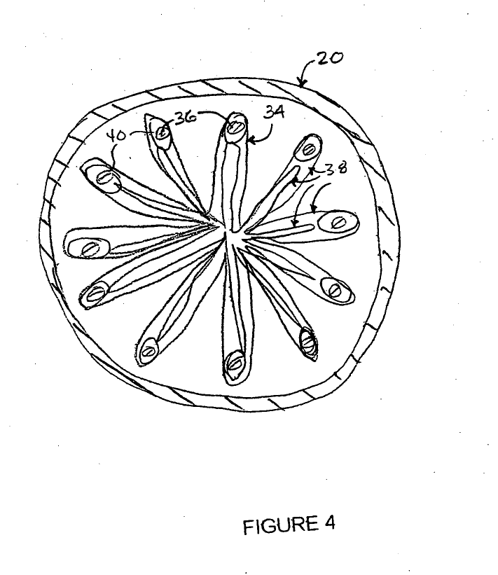

FIGS. 3 and 4 illustrate an anastomosis device 30 of the present invention folded or collapsed and retained within the catheter 20. The device 30 includes a registration means 32, such as an inflatable chamber, and a main housing 34 storing a plurality of fastening clips 36.

The main housing 34 includes foldable or flexible walls 38 and hollow rigid or semirigid elongate clip housing members or stays 40, either defined therein or secured thereto, each defining a channel and retaining a clip 36. The flexible walls 38 are formed of a sheet material, such as Dacron polyester, polytetrafluoroethylene, and the like such as GORE-TEX polytetrafluoroethylene, or other FDA class 3 materials for implantation. The elongate clips 36 are formed from a shape memory alloy (SMA) or superelastic alloy that is FDA class 3 approved for implantation, such as NITINOL or TiNi, which are nickel-titanium based alloys, or alike materials.

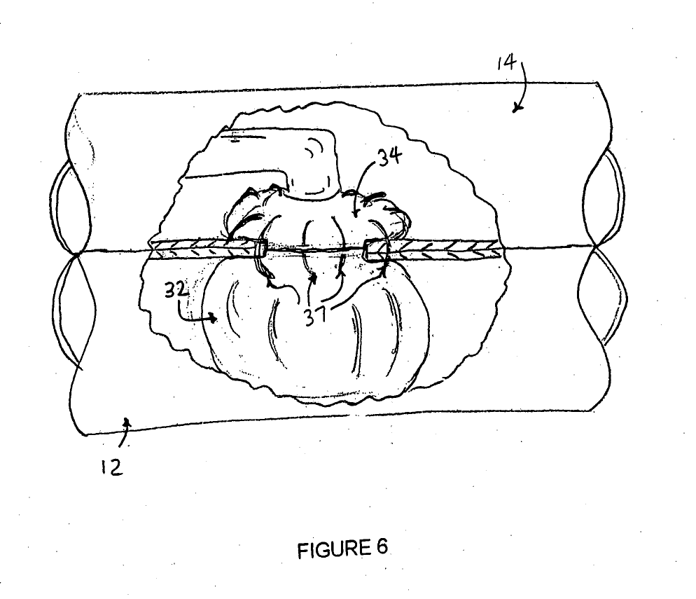

The clips are formed of a shape memory or superelastic material, e.g., a nickel-titanium alloy, for example, in the form of a wire having a needle-like point 37 (see FIG. 6) on one leading end. The shape of each clip 36 is “set” or “trained” to a curved shape in a known manner. For example, the wire can be constrained in a circular shape on a mandrel and heat-treated.

The shape-memory or superelastic clips 36 are readily deformable and are placed in the elongate channels 40, which constrain the clips in temporary, straightened shape. In a particularly preferred embodiment, the elongate channels have a slightly elliptical or oval cross-sectional shape. Since the pre-shaped clips have a lower stress when the plane of the circle defined by the unrestrained clip is aligned with the long axis of the ellipse, the clip inherently maintains the desired alignment in the channel. Thus, the orientation of the channels is selected to control the direction and orientation of the clips 36 when they are deployed. The specific preset curvature of the clips is selected to control the bite of the clip as it is deployed. Also, the alloy composition and/or the heat-treatment conditions (temperature, time) can be adjusted to impart the desired shape-memory or superelastic characteristics.

In certain embodiments, a nickel-titanium alloy designed to take advantage of the superelastic effect, i.e., having an active A_{f} temperature below the use temperature (e.g., body temperature), is employed as the clip 36 material. Such clips are extremely flexible and absorb the strain energy of being constrained in the clip housing channels 40. The strain energy is released as the applied strain is removed, i.e., when the clips are deployed from the channels 40, reverting to the helical shape. In this regard, the clip housings are elliptically shaped tubes that elastically constrain the previously shaped clips. The elliptical shape of the housing allows the direction of the deployed clip to be defined—the curvature of the clip will align itself with the major axis of the elliptical ID, which minimizes the strain in the clip. The clip may be deployed by mechanically ejection from the housing, for example, by pushing a wire (located behind the clip) into the housing. In applications where the clip remains attached to the housing (e.g., barbed attachment of patches) the clip and the pusher wire are preferably one piece (not separated) and only the helical end is exposed during deployment.

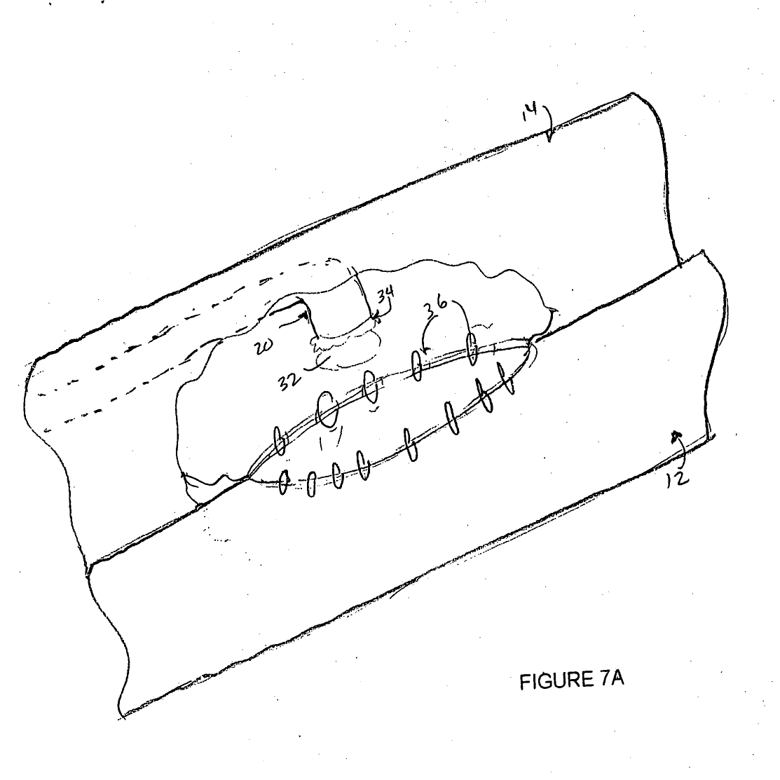

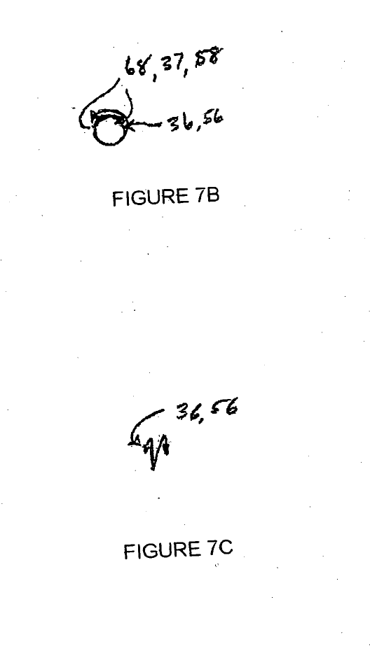

The preferred helical shape of the clips and barbs of the present invention is shown in FIGS. 7B and 7C, and is defined so that the tips 68 (also specifically identified herein as 37 and 58), of the curved clips and barbs are extended past each other sufficiently to assure that the clip remains in attachment to the desired tissue for the application of interest. The amount the tips extend beyond each other may vary depending on the medical application, the strength of the superelastic or SMA material used, the load placed on the material, the tissue in which it is deployed and its resistance to penetration. The preferred amount is thus variable within the range that permits the helical barb to be deployed to assure attachment and avoid detachment upon loading which might cause separation of the tips so that they no longer extend past each other. When extended beyond each other, the clips and barbs assure that there is always a portion of the clip in contact with tissue sought to which the clip or barb is to be secured. More than one rotation of the helix forming the helical shape is required and, typically, no more than two rotations is needed.

In other embodiments, a shape memory alloy, such as a thermally activated shape memory form of NITINOL, is employed. The clips are pliable when chilled and are readily maintained in a straightened shape in the housing channels 40. An alloy having a transformation temperature at or near body temperature is selected so that the clips will return to their circular shape when warmed to body temperature. The clip deployment is actuated by any mechanism that could exert an appropriate force on the pusher wife. For example, a toggle linkage, a pneumatic or hydraulic piston, or a shape memory spring can be used to actuate clip deployment.

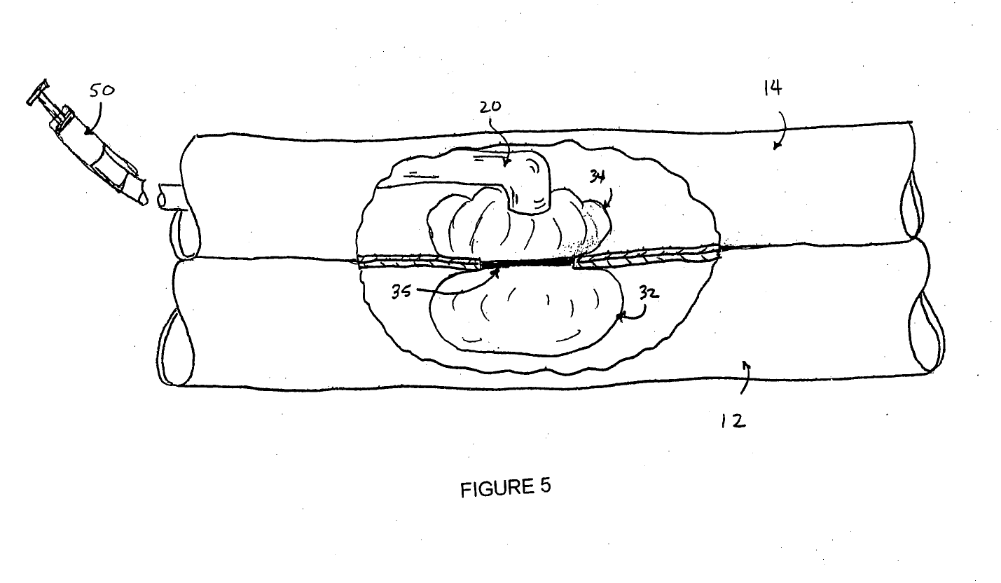

Referring now to FIG. 5, the anastomosis device 30 of the present invention is passed through the distal end 24 of the catheter 20 and is inserted through the incision 22 in the graft vessel 14 and inflated, e.g., via a syringe 50 attached at the distal end of the catheter and controlled by the operator. Other pneumatic devices for inflating the device 30 are also contemplated.

Preferably, the registration member 32 is a pneumatic chamber that, when inflated, serves to approximate and register the vessels to be. joined. The registration member 32, which is a balloon in the depicted embodiment, extends through the incision 18 and into the artery 12. The balloon 32 is shown inflated, bringing the vessels 12 and 14 together in cooperation with the housing 34 storing the clips 36. A boundary 35 between the inflatable vessels 32 and 34 serves to register the clips, while the inflated chambers 32 and 34 push the tissues between the vessels together. The boundary 35 may be, for example, a constricted region formed therein, an annular band, or the like. By providing proper alignment of the incisions, healing is facilitated. When a nonliving graft material 14 is used, the opening 22 is preferably treated to allow limited tissue ingrowth.

In an alternative embodiment (not shown), the balloon 32 is replaced with a series of mechanical fingers located between the clips, that close to clasp the tissues between the vessels, thereby accomplishing both approximation and registration functions.

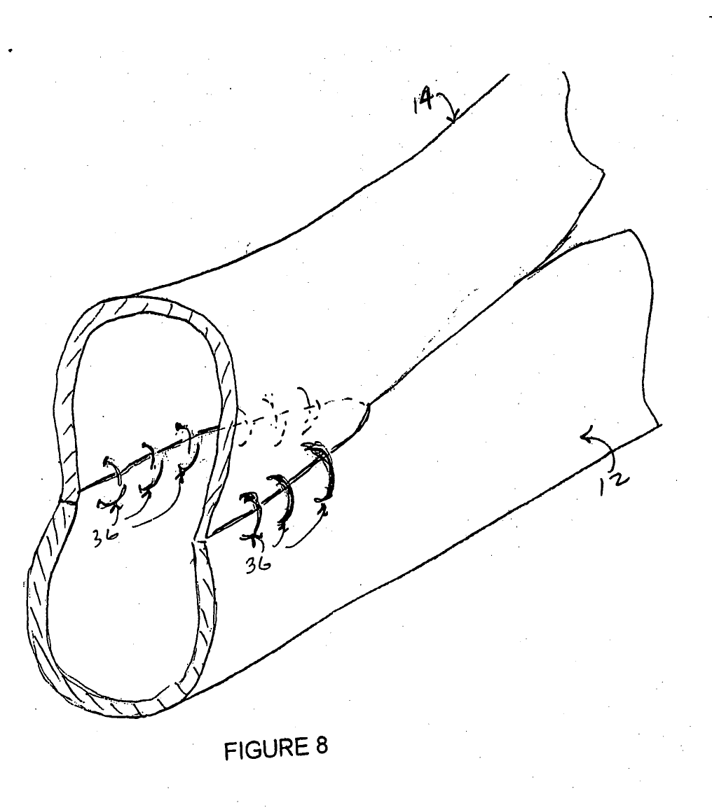

Referring now to FIG. 6, the clips 36 are deployed and the needle-like points 37 puncture and curl through the adjacent tissues of the graft 14 and artery 12 to form a strong mechanical bond therebetween. In FIGS. 7 and 8, the completed anastomosis is shown. The graft vessel 14 is shown in partial cutaway to illustrate the removal of the catheter 20, clip housing 34, and registration member 32.

In an alternative embodiment, the clips are not completely deployed from their respective housings, and one end of the clip remains attached to the housing. This barb attachment allows many different devices to be securely attached to various types of tissue. Although many other applications are appropriate, a particular application for the subject apparatus is endoventricular left ventricular (LV) volume reduction following physiologic LV remodeling (enlargement), e.g., following myocardial infarction and other heart ailments. Other applications include, for example, wound closure, attachment of biological microelectromechanical (MEMS). devices, and attaching patches for the noninvasive correction of intestinal perforations.

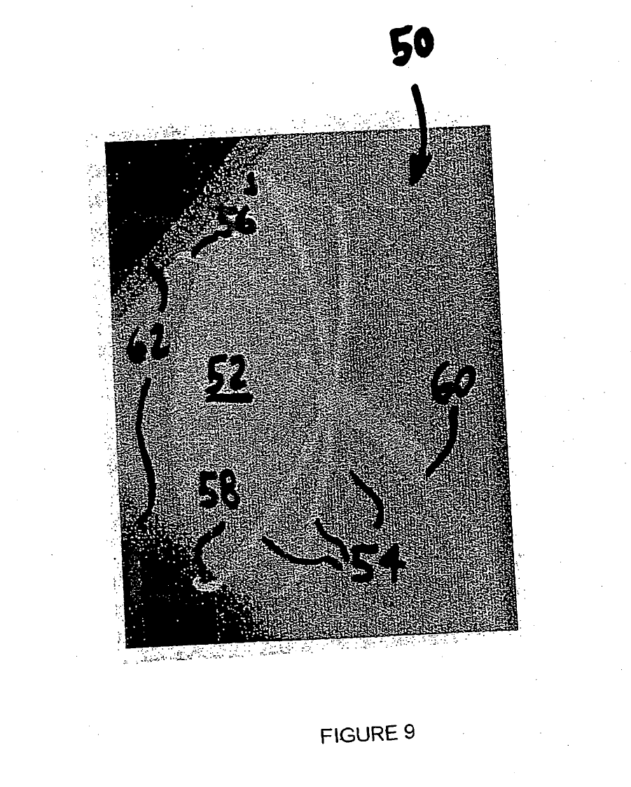

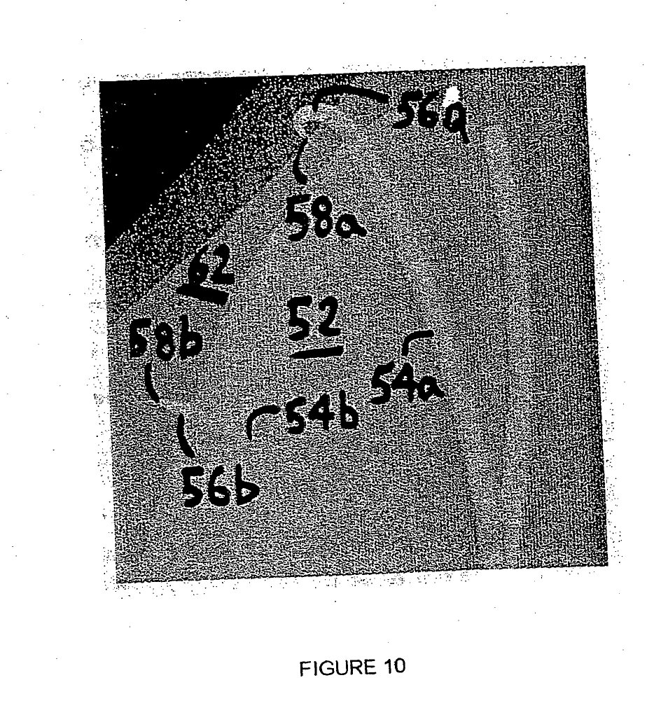

Referring now to FIGS. 9 and 10, there is shown an attachment device 50 which comprises a patch or diaphragm 52 supported by radially spaced-apart and generally rigid or semirigid hollow ribs or stays 54.

The stays 54 are hollow and each houses a barb 56. The device 50 assumes a folded or collapsed position by parallel alignment of the stays 54 and a corresponding folding of the patch material 52 for introduction into the ventricle via a catheter 60 defining a channel though which the device 50 is passed.

The patch 52 comprises a sheet material, which is circular, or, more preferably, oval or elliptical in shape. The patch may be formed from, for example, Dacron polyester, GORE-TEX polytetrafluoroethylene, or other FDA class 3 materials for implantation, which could be additionally treated with thromboses modulating agents or other prescriptions to allow controlled tissue ingrowth. Alternatively, the diaphragm can be formed of fixed mammalian tissue, such as bovine or porcine pericardium, autologous pericardium, etc.

The barbs 56 include a tissue-piercing pointed distal end 58 extending in a radially outward direction. The barbs are secured within the respective housings at an end opposite the pointed ends 58. Limited movement of the barb in the axial direction is optionally provided to extend the barbs for deployment.

The barbs 56 are formed of a nickel-titanium alloy having superelastic and/or thermally activated shape memory characteristics. When the clips are made of a superelastic alloy, the barbs are constrained in a straightened shape by the housings 54. When deployed, the barbs 56, e.g., by mechanically pushing the barbs outwardly a short distance from their respective housings, resume their helical shape, puncturing and curling into the adjacent tissue to form a strong mechanical connection. Similarly, a shape memory alloy which becomes activated at or below body-temperature can be used in similar fashion, in which case the thermally activated alloy is cooled to increase its flexibility and returns to the trained helical shape upon warming within the body. Each barb may be ejected as described above, for example, using a pusher wire urging, or more preferably, attached to the proximal end of the barb, and so forth.

In an especially preferred embodiment, the barbs are deployed sequentially to generate a torque that helps to assure contact of the diaphragm with the adjacent tissue 62, such as an inner ventricular surface. The sequential deployment is illustrated in FIG. 10. A first barb 56a having a pointed end 58a extends from within housing 54a in its fully deployed position and a second barb 56b having a pointed end 58b is just starting to project into the myocardium 62. The barbs are sequentially deployed (counterclockwise in the depicted illustration) until all of the barbs have been deployed. The cross-sectional shape of the sleeves 54 controls the orientation of the barbs. As described above, the barb housings 54 have an oval or other cross-sectional shape providing a preferred orientation of the barbs 58.

As may be further understood from the discussion above, the present invention has further, broader application to a wide variety of medical environments within the body to secure patches or to suture a site requiring closure, such as needed for wound repair, deep hole wound closures, intestinal perforations, incision sites and other needs where closure or suturing opposing tissues at a site are required.

The method for repairing tissue at a site requiring closure includes first introducing a patch into the body, preferably through a non-invasive catheter procedure. Such procedures may be initiated through vessels of various types, or initiated through the body cavity. The catheter would carry a patch or sheet of biologically compatible material, adapted for the specific tissue targeted for repair. Materials appropriate for closure of different sites in the body are known in the art for exposure to the environments in which they must function, and those discussed above are illustrative for the cardiovascular environment.

As with the illustrative device shown in FIGS. 9 and 10, the sheet of biocompatible material preferably includes a plurality of generally rigid elongate sleeves. Such sleeves may either be present in the sheet or attached to the sheet, so long as they can function to permit movement of a surgical barb therefrom. The sleeves are positioned to extend the sheet of biocompatible material into a configuration suitable to enclose the selected portion of the body as a patch.

The patch is then secured over the portion requiring closure by extending said barbs to a helical position so that the barbs engage tissue surrounding the portion requiring closure. The barbs are made of materials as described above, such as superelastic, shape memory materials, or like materials, and are preferably extendable using a mechanical device to push them partially from the sleeves, in a manner similar to that described above, to deploy to a helical position. It is understood, however that the barbs may be activated assume a helical shape in other ways, including electrical heating, ultrasonic activation from a remote source, and timing devices. In accordance with the invention, the barbs are extended to a helical shape to provide for attachment of the patch without further suturing, and enable the patch to be attached from one side of the site, without further intrusion into the targeted site.

The present invention further provides for the suturing of tissue at a site requiring closure using the helical barbs. The method again preferably involves the introduction of elongate sleeves, preferably through a catheter or other non-invasive tool. Surgical barbs are movably secured within each sleeve, and are spaced apart as desired for the suturing procedure desired. In the extended position, the surgical barbs will return to a helical shape, whether activated by temperature, electrical current, external power supply or a timer circuit to trigger conditions for shape change.

In the suturing application, the sleeves with barbs may be positioned at the site requiring closure one at a time, or in groups, or as a cluster attached to a sheet that deploys them to a desired spacing, whether such spacing is parallel, radial or in otherwise oriented. After positioning the sleeve or sleeves into the desired position, the barb is extended from the sleeve into a predefined helical position where the barb engages tissue on opposing tissues at the site requiring closure and serves as a suture. The helical barb is preferably then fully expelled from the sleeve, and the sleeve removed from the body or used to deliver consecutive barbs. When functioning as a remotely positioned suture, the helical barb of the present invention has the distinct advantage of providing secure connection from just one side of a tissue, without needing access to the opposing side of the tissue to achieve a secure attachment. It further serves both as the needle and the suture, and may be made of materials as discussed herein.

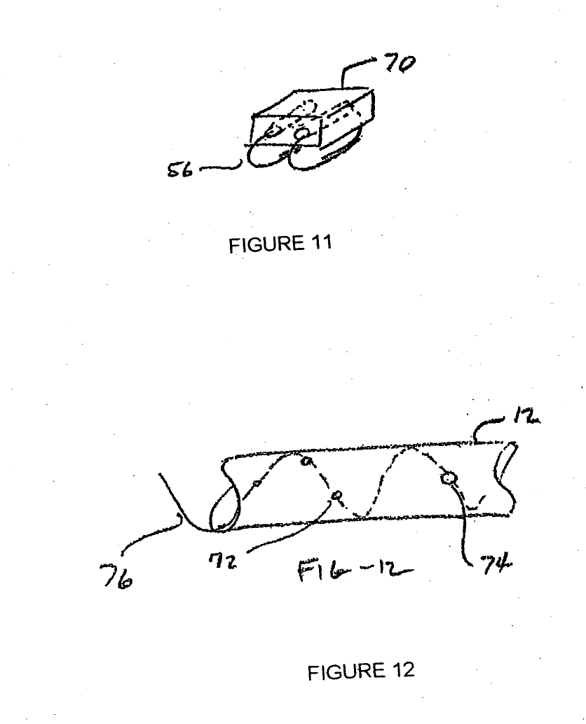

In a still further application in the medical field, the present invention encompasses a helical barb device that is suitable for affixing medical devices, generally, to tissues. While illustratively shown in the process of attaching a diagram in FIGS. 9 and 10 or securing a graft to an artery in FIGS. 1 to 6, the present invention may further be used to attach biological microelectromechanical systems (MEMS), sensors, filters, batteries or other implantable devices 70 as shown representatively in FIG. 11. Again, the helical barb has the distinct advantage of providing secure connection from one side of a tissue, without needing access to the opposing side of the tissue to achieve a secure attachment. It further serves both as the needle and the suture, and may be made of materials as discussed herein.

Where the helical barb affixes medical devices within the body, it may be deployed in a manner similar to that described above from a sleeve attached to a larger implantable device, where a portion of the barb remains attached within the sleeve. Alternatively, the sleeve might also serve as the device to carry a device, whose inner and outer surface are exposed to the targeted tissues upon deployment of the helical barb.

Where micro and nanotechnologies are being inserted into the body, the helical barb itself may be the carrier for small devices attached to the surface of the barb. In this configuration, the micro or nano devices are appropriately positioned on the barb when it is in its first, non-deployed position, such as an elongated position in a sleeve, so that in its second position penetrating and attaching to tissue, the devices would be presented to the tissue without damage, and in a position where they function as desired. The devices so implanted are contemplated to monitor or measure biological conditions, manufacture or deliver medically efficacious materials, or perform other desired device functions. Where the helical barb serves as a carrier for devices, it is preferred that they be deployed in a manner similar to that described above for a sutures, separating from the delivery mechanism.

In its application to attach a device to tissue in a medical procedure, the helical barb functions to connect a man-made, preferably biologically compatible material to a biological material, in contrast to the suturing of two biological materials. By way of example and not limitation, devices which may be attached using the helical barb of the present invention include blood pressure transducers, glucose monitors, fluid flow sensors to detect bleeding at operative sites, leaking aneurysms, or local fluid build-up.

The provision of a simple helical barb means to deliver a MEMs device, or other nano or micro technology makes possible the dream of drug delivery at the site of need using a device which will remain in place where chosen. The potential to use such devices as small local drug factories, or as drug dispensing agents from which drugs can disperse over time is known in the art. The helical barb device of the present invention provides a secure means of attachment, whether serving as an attachment means for a larger device or as the carrier for very small drug delivery devices.

It is further contemplated in the drug delivery application of the present invention that the material of the helical barb may be hollow, and serve as a carrier for drugs. The hollow ends may be plugged with biodegradable material, or simply be plugged by the delivery mechanism so that once fully deployed and released at least one end of the helical barb becomes exposed to deliver treatment materials as the delivery device retracts. Alternatively, the helical barb may be coated with medicament, so that it is delivered directly to the tissue upon the penetration and attachment of the barb.

It is still further contemplated that the helical barb of the present invention may include biodegradable materials whose period of integrity is designed to last as long as the drug delivery device remains operable, or as long as timed release of drugs from a device lasts, after which time the helical barb device is designed to fail so that the device may be easily released and either expelled or retrieved from the body.

Medically efficacious materials that may be delivered include drugs, hormones, small molecules, proteins, genetic materials, radioactive materials, markers, biological agents and other treatment materials. By way of example and not limitation, human growth factor Veg-F might be delivered using the helical barb or in combination with a MEMs or other devices to treat coronary artery blockage, promote angiogenisis, or provide cardiac drug delivery without the need for repeated hearth catheterizations. Use in targeted high blood flow areas can also provide for enhanced treatment opportunities. Radioisotopes and chemotherapeutic agents may also be placed and positively attached with the helical barb of the present invention directly at the site of tumors, providing longer-term drug delivery.

In a further drug delivery application, the helical barb of the present invention (in a first elongated position), an implantable device, or a the sleeve may be initially implanted at a desired biological location by coating at least a portion thereof with a biological/protein attachment material targeting the desired site. Numerous targeting proteins are discussed in the art for different functional organs. In one configuration, the helical barb may be treated in its first elongate position, ingested or implanted, separately or in combination with a device, and once the device or barb adheres chemically to the targeted portion of the body, the attachment may then be made more permanent by delayed deployment of the helical barbs.

The helical barbs may be deployed in such an application, as with the other applications of the present invention discussed herein, through timed action or remote signal, such as an ultrasonic signal or other trigger, electrical current, temperature, or timed release of the barbs from a device.

In addition to serving as a helical barb for attachment, the helical device of the present invention may serve to lodge itself in a vessel or other generally tubular structure or opening by deployment from a first position, preferably by means of a catheter, to a second helical position where the helical device relies on its radius of curvature to expand to a size at which it lodges in the vessel, or other biological structure, opening, duct or orifice.

Such a vessel, structure, opening or duct would have an average radius less than that of the helical device. As shown in FIG. 12, once in this position, the helical device may serve as a stent 76, as a carrier for a device 70, such as a sensor 74 or MEMs 72 as described above, or as a drug delivery vehicle as described above.

When serving as a stent, the helical device may be configured to have many loops, as shown in FIG. 12 rather than the preferred design of the helical barb shown in FIG. 7B and described above. Further, in a stent, MEMs devices may be embedded in the superelastic or shape memory material, or spaced on the surface for monitoring or measuring of biological or treatment parameters or for drug delivery, or combinations thereof.

The helical barbs, fastening clips and helical devices of the present invention are preferably formed either of a shape memory alloy (SMA) or a superelastic alloy that is FDA class 3 approved for implantation, such as NITINOL or TiNi, which are nickel-titanium based alloys, or alike materials, and are formed as a wire with diameters down to 25 microns, and have a needle-like point on at least one end. Some polymers, including but not limited to starch-based polymers, are also known which exhibit superelastic properties desirable for application in accordance with the various aspects of the invention described herein. In addition, the ends of the barbs may include end treatments such as barbs and teeth that provide additional anchoring capability for the barbs, devices, sutures and attachments described herein.

Use of SMA materials is contemplated for the above applications. Among the opportunities provided by such materials is the design of the shape-memory transition temperature. so that the natural hysteresis between phases would advantageously be set to enable activation of the material to cause shape change by electrical means. Specifically, the high transition to austentite would be just above body temperature. An electric current provide or induced in an adjacent or attached device would provide heat for the shape memory triggering once the helical barb was in position at the desired site for attachment to tissue or deployment into a helical shape. As long as the lower transition back to martinsite is below body temperature, the helical barb will hold its shape, just as with a device prepared for deployment upon exposure to body temperature.

In addition, the hysteresis of shape memory materials provides the opportunity for removal of devices after placement. If the memory shape is triggered by a temperature slightly higher than the body temperatures, then a return to a temperature slightly lower than body temperature, by using catheter supplied cooling, such as cooled fluids, cryogenic probes, cooled heat sinks, heat pipes, thermoelectric or other cooling devices, may provide for easier device removal.

The invention has been described with reference to the preferred embodiment. Obviously, modifications and alterations will occur to others, upon reading and understanding the proceeding detailed description.

It is intended that the invention be construed as including all such modifications and alterations insofar as they come within the scope of the appended claims or the equivalents thereof.

1. A method for delivering a medical treatment to a living organism comprising:
providing at least one attachment device;
deploying at least one said attachment device to a generally helical shape inside with said living organism; and
producing a medically significant effect related to said attachment device.

2. A method of claim 1, wherein:
said attachment device is a helical carrier device including a medically efficacious substance;
said step of deploying comprises penetrating and attaching said helical carrier device to tissue inside said living organism; and
said step of producing a medically significant effect comprises dispensing a medically efficacious substance from said helical carrier device.

3. The method of claim 2, wherein
said step of providing said attachment device comprises
treating said helical carrier device with a substance chosen to target a given location in the body; and
providing the helical carrier device in a first position;
said step of deploying includes:
ingesting said helical carrier device when in a first position; and
activating said helical carrier device to deform to a second helical position and attach to tissue after reaching the targeted location in the body.

4. The method of claim 1, wherein:
said step of providing an attachment device comprises
providing a helical carrier device comprised of a material having a first shape permitting insertion into the body, and a second helical shape having a radius of curvature that is larger than the average radial dimension of the biological material in which it is disposed, wherein said helical carrier device further includes a medically efficacious substance;
said step of deploying comprises:
inserting said helical carrier device into a biological material in the body while said helical carrier is in a first position; and
exposing said helical carrier device to conditions causing said helical carrier device to assume said second, helical shape larger than the average radial dimension of the biological material in which it is disposed; and
said step of producing a medically significant effect comprises dispensing a medically efficacious substance from said helical carrier device.

5. The method of claim 1, wherein:
said step of providing an attachment device comprises providing an elongate material having a first shape suitable for insertion into a tubular biological structure in a living organism; and
said step of deploying comprises exposing said elongate material to conditions causing said elongate material to assume a second helical shape having a radius of curvature a larger than the average radial dimension of the biological material in which it is disposed.

6. The method of claim 1, further comprising:
providing a registration device;
providing a catheter; and
providing a tissue fastening apparatus, said tissue fastening apparatus including:
a housing member having a plurality of elongate channels defined therein, the housing member being movable between a first collapsed position and a second expanded position, wherein ones of said attachment devices are positioned in ones of said channels; and
an attachment device deployment mechanism for projecting said attachment devices from their respective channels; and
wherein said step of deploying comprises:
forming a first elongate incision in a vascular graft;
forming a second elongate incision in a target coronary artery of the heart, the first and second incisions defining an anastomotic site;
inserting said catheter into the graft;
aligning the graft and target artery so that the first and second incisions are in aligned, facing relation;
passing said registration device from the catheter through the first and second incisions and into the target artery;
passing said tissue fastening apparatus from the catheter into the graft:
approximating the graft and target artery using said registration device;
deploying the attachment devices from their respective channels using the attachment device deployment mechanism; and
removing the housing member, deployment mechanism, registration device, and catheter from the anastomotic site.

7. The method of claim 1, further comprising:
introducing a patch into the ventricle of the heart of said living organism, the patch including:
a sheet of biocompatible material adapted for placement within a ventricle;
a plurality of generally rigid elongate sleeves attached to the sheet, the sleeves spaced apart and extending radially; and
wherein
said step of providing comprises providing ones of said attachment devices in ones of said sleeves, said attachment devices movably disposed within each sleeve; and
said step of deploying comprises securing the patch to an interior wall of the ventricle by moving said attachment devices into engagement with said ventricle and into a generally helical shape;
said step of producing comprises reducing the volume of the ventricle.

8. The method of claim 1, further comprising:
introducing a patch into the body of said organism, the patch including:
a sheet of biocompatible material adapted for placement within a selected portion of the body;
a plurality of generally rigid elongate sleeves attached to the sheet, the sleeves positioned to extend the sheet of biocompatible material into a configuration suitable to enclose a selected portion of the body as a patch, wherein ones of said attachment device are movably disposed within ones of said sleeves; and
wherein:
said step of deploying comprises extending said attachment devices to a helical position where said attachment devices engage tissue surrounding the selected portion of the body; and
said step of producing comprises enclosing said selected portion of the body with said patch.

9. The method of claim 1, wherein:
said step of providing an attachment device includes:
providing at least one generally rigid elongate sleeve wherein ones of said attachment device are movably secured within each sleeve;
said step of deploying comprises:
positioning said sleeves into a desired configuration in proximity to two portions of tissue in the body of said organism; and
extending at least one of said attachment devices from said sleeves into a predefined helical position where said attachment devices engage said two portions of tissue;
said step of producing a medically significant effect comprises connecting said two portions of tissue.

10. The method of claim 1, wherein:
said step of deploying comprises:
deploying said attachment device from a single side of a first material; and
penetrating a second material with said attachment device; and
said step of producing a medically significant effect comprises connecting said first and second materials together.

11. The method of claim 10, wherein:
said first material is a medical device and said second material is tissue in the body of the organism,
whereby a medical device is secured to said tissue by access to only a single side of the biological material.

12. The method of claim 1, wherein said attachment device includes a medical device.

13. A tissue fastening apparatus comprising an attachment device deployable from a single side of a biological material.

14. The tissue fastening apparatus of claim 13, where said attachment device is deployable into a generally helical shape.

15. The tissue fastening apparatus of claim 13, wherein the attachment device is formed from a material selected from the group comprising: superelastic material, thermally activated shape memory material, and combinations thereof.

16. The tissue fastening apparatus of claim 13, wherein said attachment device:
is ingestible when in a first position,
is treated with a substance chosen to target a given location in the body, and
further comprises an activation trigger to cause said attachment device to deform and deploy into the biological material after reaching the targeted location in the body.

17. The tissue fastening apparatus of claim 13, further including at least one from the group of materials having medical significance, comprising: a medicament, a biologically active material, genetic material, proteins, radioactive materials and combinations thereof.

18. The tissue fastening apparatus of claim 13, further including a medical device.

19. The tissue fastening apparatus of claim 18, wherein said medical device includes one from the group of devices comprising: biological manufacturing devices, pharmaceutical manufacturing devices, electronic devices, electrical devices, power sources, mechanical devices, micromechanical devices, microelectromechanical devices, monitoring devices, sampling devices and combinations thereof.

20. The tissue fastening apparatus of claim 13, wherein:
the attachment device is comprised of a material having a first shape permitting insertion inside the inner surface of a generally tubular biological material in the body, and
the attachment device is deployable into a second generally helical shape having a radius of curvature that is larger than the average radial dimension of the generally tubular biological material in which it is disposed.

21. The tissue fastening apparatus of claim 13, further comprising:
a housing member having a plurality of elongate channels defined therein;
the housing member being movable between a first collapsed position and a second expanded position; and
a plurality of said attachment devices,
wherein ones of said attachment devices are positioned in ones of said channels for deployment from a first position in said channels to a second generally helical position.

22. The tissue fastening apparatus of claim 21, further comprising:
at least one deployment mechanism for projecting at least one of said attachment devices from its respective housing member; and
a registration member;
whereby said housing member in said first collapsed position may be inserted into a first tubular structure, said registration member may align said first and second tubular structures, and said housing member in said second expanded position may deploy ones of said attachment devices to connect the first and second tubular structures.

23. The tissue fastening apparatus of claim 22, wherein the attachment devices are formed from a material selected from a superelastic material and a thermally activated shape memory material.

24. The tissue fastening apparatus of claim 13, further comprising:
a sheet of biocompatible material adapted for placement within a targeted tissue;
a plurality of generally rigid elongate sleeves attached to the sheet, the sleeves spaced apart and extending generally radially; and
a plurality of said attachment devices, ones of said attachment devices movably secured within ones of said sleeve, movable between a first constrained position and a second generally helical position such that said sheet is in fastened relationship to a surface of the targeted tissue.
